(19) United States
(12) Patent Application Publication
(10) Pub. No.: US 2002/0138898 A1
(43) Pub. Date: Oct. 3, 2002

(54) Dual single-eye swimming goggles system

(76) Inventor: Herman Chiang, Chung-Ho City (TW)

Correspondence Address: Keith Kline, PRO-TECHTOR INTERNATIONAL SERVICES, 20775 Norada Court, Saratoga, CA, 95070-3018, US

(21) Appl. No.: 09/825,213

(22) Filed: Apr. 2, 2001

(51) Int. Cl. A61F 9/02

(52) U.S. Cl. 2/428

(57) ABSTRACT

A dual single-eye swimming goggles system for swimming pool purposes comprising: a first single-eye swimming goggle and a second single-eye swimming goggle as well as a method for being worn single-eye swimming goggles on a wearer's head, each single-eye swimming goggles having a lens frame with a lens fixed therein, and a head strap mean mounted to the lens frame, each single-eye swimming goggle can respectively adjusting to match the periphery of a wearer's eyehole, so as to both eye of a user get more comfortable during use the dual single-eye swimming goggles; beside, to prevent consequently the seepage of water.

### FIELD OF THE INVENTION

The present invention relates to a swimming goggles and particularly to a dual single-eye swimming goggles for being worn on the periphery of eyehole respectively, to conform with a wearer's eyehole contour, and to provide more secure and comfortable fitting when the dual single-eye swimming goggles are in use.

### BACKGROUND OF THE INVENTION

Conventionally, a swimming goggles has an adjustment element for adjusting a span between a left lens frame and a right lens frame. This is so because the span between two eyes as well as the eyehole's contour may be different from person to person. In addition, the swimming goggles has gaskets for contacting with periphery of a wearer's eyehole, in order to get comfortable and prevent the seepage of water

However, the left lens frame and the right lens frame always connect together by a bridge in all types of swimming goggles, which makes the left lens frame and the right lens frame influence each other when adjusting a span of the bridge In other words, adjusting one of the lenses frame to be in conformity with the periphery of left eyehole or right eyehole will move the other lenses frame during the adjustment of span after the swimming goggles is worn on the wearer's eyeholes. Hence, the left lens frame and right lens frame never respectively match wearer's eyehole contour since the left lens fame and the right lens frame influence each other during the adjustment of span, thereby resulting in the lens frame being not able to seal up the periphery of each eyehole and leading to the formation of a gap between each of the lens frames and the seepage of water when swimming goggles are in use.

### OBJECTS OF THE INVENTION

Therefore, the object of the present invention is to provide a dual single-eye swimming goggles system that can overcome the drawback of the aforementioned prior art.

More specifically, the main object of the present invention is to provide a dual single-eye swimming goggles system which respectively conform with the periphery of each eyehole of wearer when each single-eye swimming goggle in use, and which never influence the adjustment of span of each single-eye swimming goggle each other, to provide securely matching with the periphery of eyehole respectively and get more comfortable and effective fitting when the dual single-eye swimming goggles are in use.

Another object of the present invention is provide to a dual single-eye swimming goggles which has a method for wearing each single-eye swimming goggle on a periphery of a wearer's eyeholes respectively, this way differ from the prior art which wear both lens frame of a swimming goggles on a wearer's eyeholes together. Thereby resulting in each single-eye swimming goggles can seal up the periphery of a wearer's eyehole and prevent the seepage of water when in use.

To achieve the above objects, the present invention is featured in that a dual single-eye swimming goggles system have a first single-eye swimming goggle and a second single-eye swimming goggle which respectively wear on a wearer's periphery of eyehole, each single-eye swimming goggles comprises a lens frame with a lens fixed therein, and a head strap mean is mounted to the lens frame.

According to the above features of the present invention, the lens frame has a groove formed in the periphery of outboard, In addition, The head strap mean comprising a strap and a fastener, wherein the strap has a receiving hole which be coupling to the groove of the lens frame, and has two free end which pass through and position the fastener.

In an aspect of the present invention, between the head strap mean of each single-eye swimming goggles has a connection element which link each head strap means together.

The another feature of the present invention is to provide a dual single-eye swimming goggles system having a method for wearing each single-eye swimming goggles on wearer's head, comprising a first step and a second step respectively wear each single-eye swimming goggle on the periphery of a wearer's eye-hole, and having an adjustment step for adjusting each single-eye swimming goggle conform with the periphery of a wearer's eye-hole respectively

According to another feature of the present invention, the adjustment step including respectively adjust each strap and single-eye swimming goggles after first step and second step are processed.

### BRIEF DESCRIPTION OF THE DRAWINGS

Other features and advantages of the present invention will become apparent in the following detailed description of the preferred embodiments with reference to the accompanying drawings of which:

FIG. 1 is a perspective view of the disassembled parts of the dual single-eye swimming goggles of the present invention;

FIG. 2 is a perspective view of the dual single-eye swimming goggles of the present invention;

FIG. 3 is a cross-sectional view of the A-A section of FIG. 2;

FIG. 4 is flow sheet view of the method which wear the dual single-eye swimming goggles system of the present invention;

FIG. 5,6 is a front view of the dual single-eye swimming goggle with a wearing step embodiment of the present invention.

### DETAILED DESCIRIPTIION OF THE PREFERRED EMBODIMENT

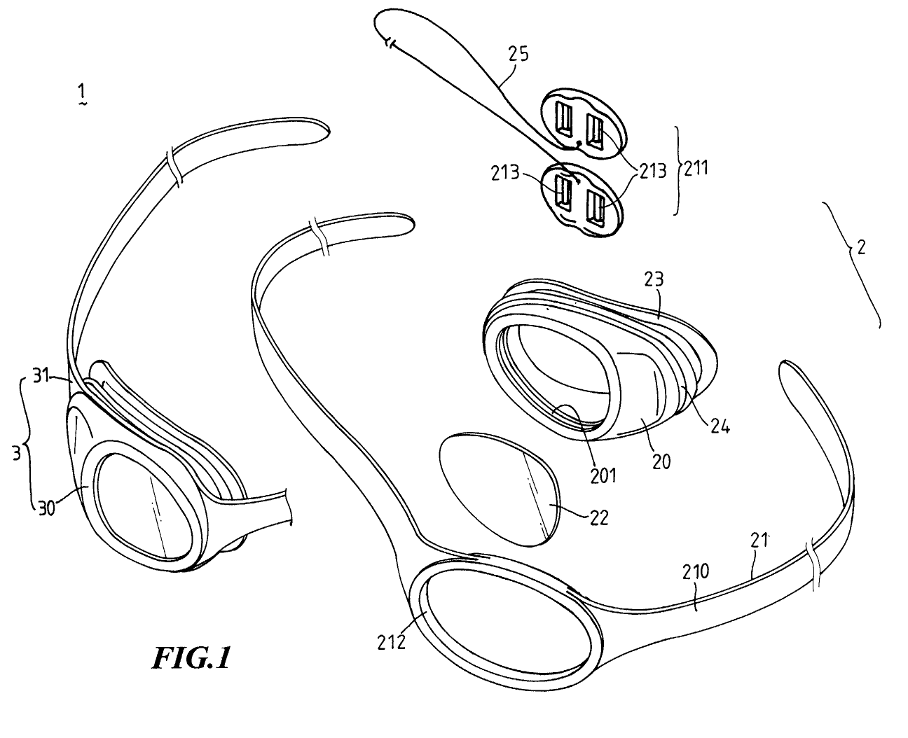

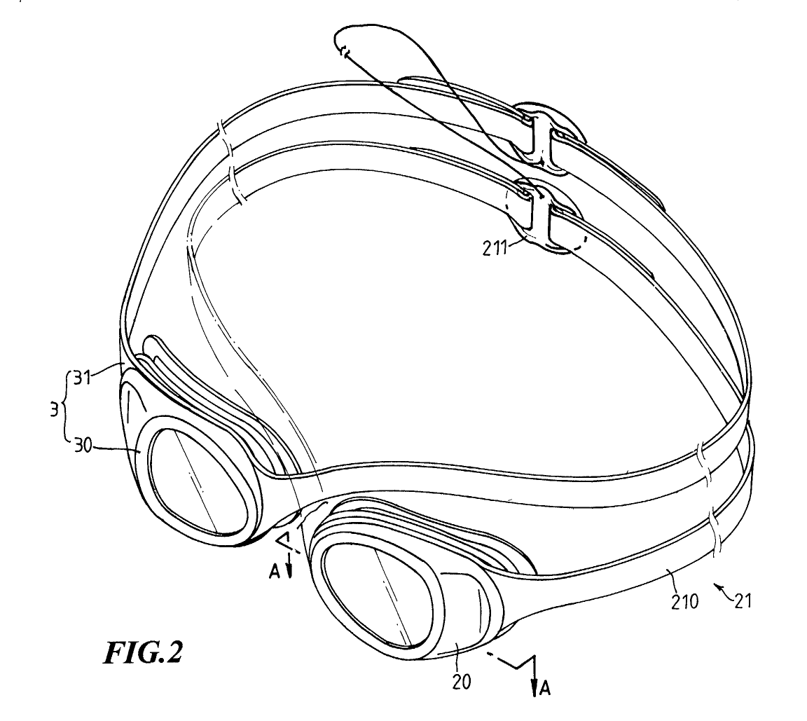

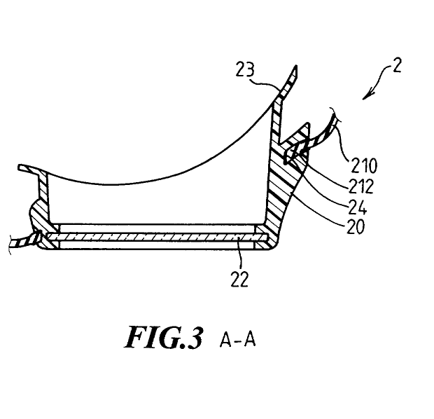

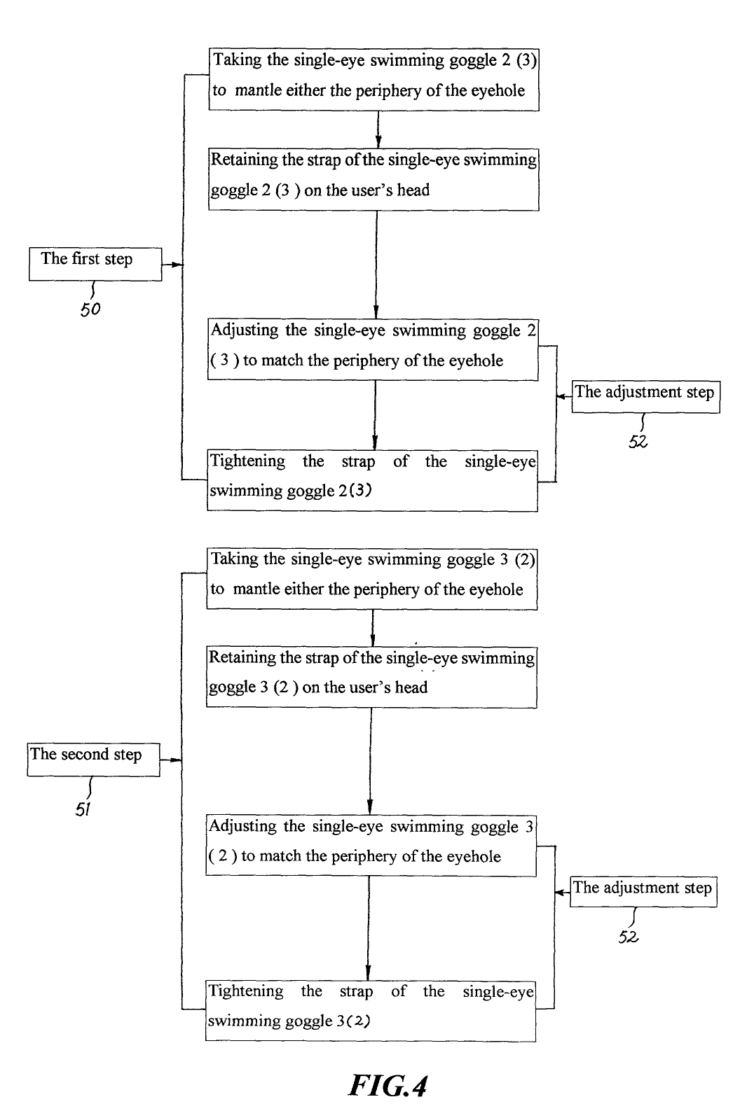

Referring to FIG. 1, the preferred embodiment of a dual single-eye swimming goggles according to the present invention is show to comprise a first single-eye swimming goggle 2 and a second single-eye swimming goggle 3 and a method for wearing each single-eye swimming goggles 2,3, wherein each single-eye swimming goggles 2,3 including a lens frame 20,30 and a head strap mean 21,31. The first single-eye swimming goggle 2 and the second single-eye swimming goggle 3 is a same conformation which show every parts of detail by the single-eye swimming goggle 2, and have assembled the single-eye swimming goggle 3 as in FIG. 1. Here just introduces the disassembled of single-eye swimming goggle 2 in order to explain convenient to understanding,. The lens frame 20 of single-eye swimming goggle 2 has an inner periphery 201 that defines a lens units 22 retaining space as in FIG. 1, a gasket 23 is formed integrally with the lens frame 20 and far away from the lens units 22 for providing touch a periphery of eyehole, and a groove 24 is located nearby the gasket 23 for severing to the head strap mean 21 position. The head strap mean 21 comprise a strap 210and a fastener 211, wherein the strap 210 has a receiving hole 212 which be coupled to the groove 24 of the lens frame 20. The fastener 211 is generally a tab and have two through hole 213 located to both a side of the fastener 211 respectively for passing through and position two free end of the strap 210. In addition, a connection element is a flexible thread 25 and have two end which are respectively disposed to between the fastener 211 of the single-eye swimming goggle 2 and single-eye swimming goggle 3 for connecting the single-eye swimming goggle 2 and the single-eye swimming goggle 3 together, the thread 25 is 10 cm in this embodiment of the present invention. With reference to FIG. 2.is a perspective view of the dual single-eye swimming goggles of the present invention. During assembly, the lens units 22 is received the inner periphery 201 firstly, then the receiving hole 212 of the strap 210 is coupled to the groove 24 of the lens frame 20 as shown in FIG.3, finally, two free end of the strap 210 are respectively passing through both the through hole2l3 of the fastener 211, the single-eye swimming goggle 2 is ready for use at this time.

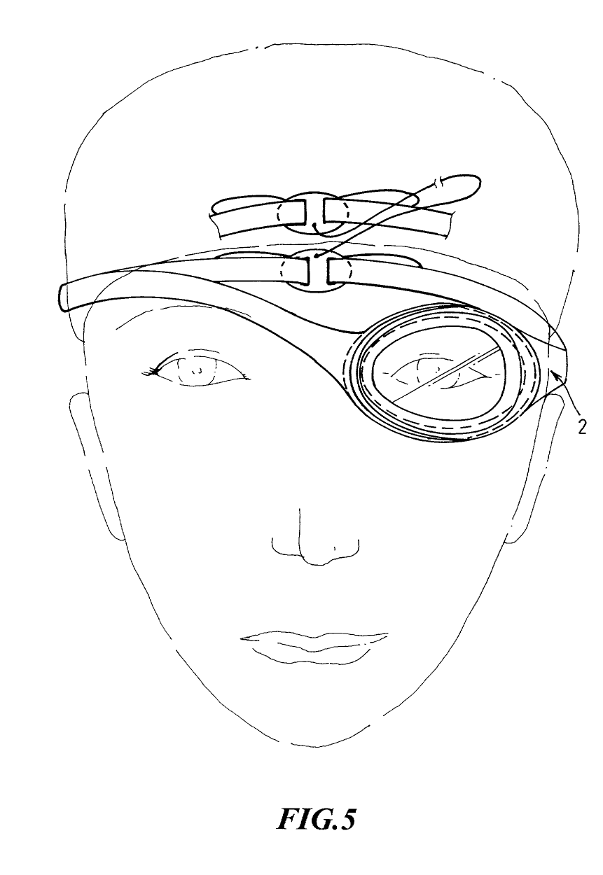

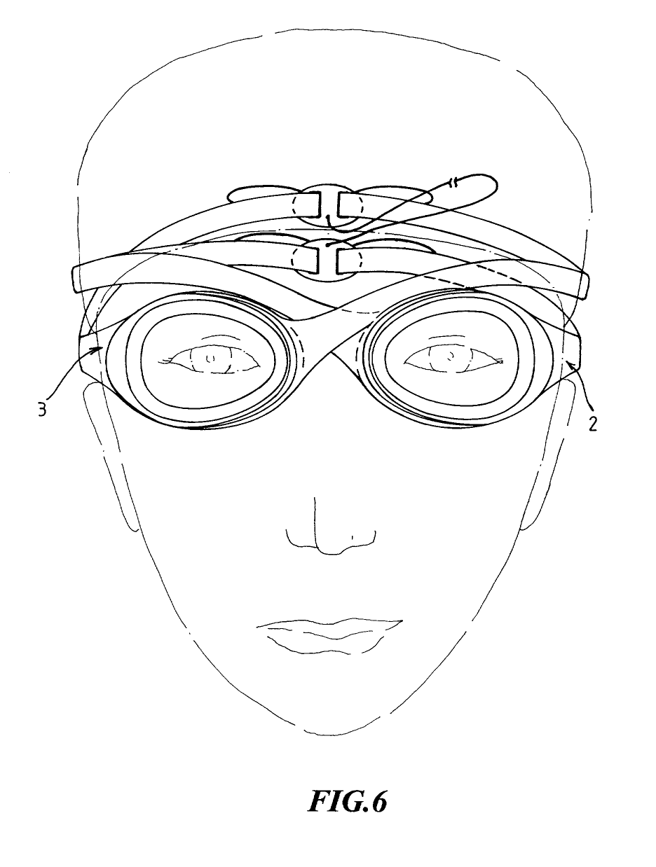

Referring to FIG. 4,5 and 6, the method of wearing for the dual single-eye swimming goggles of the present invention comprising a first step 50, a second step 51 and an adjusting step 52 as shown in FIG.4, wherein the first step 50 included taking the first single-eye swimming goggle 2 (or the second single-eye swimming goggle 3) to mantle either the periphery of the eyehole as shown in FIG. 5, then being to retain the strap 210 of the first single-eye swimming goggle 2 (or the second single-eye swimming goggle 3 ) on the user's head and being to adjust the first single-eye swimming goggle 2 ( or the second single-eye swimming goggle 3 ) to match the periphery of the eyehole, finally, the strap 210 of the first single-eye swimming goggle 2 ( or the second single-eye swimming goggle 3 ) via the adjustment step 52 adjusting which be varied and posited by adjusting the length of the strap 210 between the through hole 213of the fastener 211, so as to be quite conformity with a wearer's single-eye contour and get more comfortable for contracting of a face. The second step 51 is generally similar to the first step 50 which be retaining the single-eye swimming goggle 3 (or the single-eye swimming goggle 2 ) on the user's the other eye. Afterwards, both the single-eye swimming goggle 2 and 3 are worn around the user's head. Quite obviously, the dual single-eye swimming goggles system in accordance with the present invention comprises two single-eye swimming goggle 2 and 3 which can respectively adjusting each single-eye swimming goggles to match each the periphery of the eyehole, so as to both eye of a user get more comfortable during use the dual single-eye swimming goggles; beside, to prevent consequently the seepage of water.

While the present invention has been described in connection with what is considered the most practical and preferred embodiment, it is understood that this invention is not limited to the disclosed embodiments but is intended to cover various arrangements, which a head strap is integrally formed with each lens frame respectively, or to change the way of wearing the dual single-eye on the eyehole in use, included within the spirit and scope of the broadest interpretation so as to encompass all such modifications and equivalent arrangements.

What is claimed is:

1. A dual single-eye swimming goggles system comprising a first single-eye swimming goggle and a second single-eye swimming goggle for being worn on a wearer's head, each single-eye swimming goggles having a lens frame, a lens fixed in the lens frame, and a head strap mean mounted to the lens frame, each single-eye swimming goggles being adjustable independently of the single-eye swimming goggles to match the periphery of a wearer's eyehole.

2. The dual single-eye swimming goggles system as claimed in claim 1, wherein the lens frame has a groove formed in an out periphery thereof and the head strap mean comprises a strap and a fastener, the strap having a receiving hole coupled to the groove of the lens frame, and two free ends passing through the fastener.

3. The dual single-eye swimming goggles system as claimed in claim 2, wherein the fastener of the head strap mean is a tab having at least two through holes on opposite sides of the tab for the strap to pass through.

4. The dual single-eye swimming goggles as claimed in claim 1, further comprising a connection element which link the two head strap means together.

5. The dual single-eye swimming goggles system as claimed in claim 4, wherein the connection element is a flexible thread having two ends which are respectively disposed on the fastener of each head strap means.

6. The dual single-eye swimming goggles system as claimed in claim 1, wherein the lens frame of each single-eye swimming goggles has a gasket which is located near a contacting face side of the lens frame.

7. The dual single-eye swimming goggles system as claimed in claim 6, wherein the gasket is formed integrally with the lens frame.

8. A method of wearing a swimming goggles comprising: a first step and a second step of respectively wearing a first and a second single-eye swimming goggle on a respective periphery of a wearer's eye-holes, and an adjustment step of independently adjusting each single-eye swimming goggle in conformity with the periphery of eye-hole.

9. The method as claimed in claim 8,wherein the first step and the second step is a same way that taking the first single-eye swimming goggle (or the second single-eye swimming goggle ) to mantle either the periphery of the eyehole respectively, then to retain the strap of the first single-eye swimming goggle (or the second single-eye swimming goggle ) on the user's head and adjusting the first single-eye swimming goggle ( or the second single-eye swimming goggle ) to match the periphery of the eyehole, finally, the first single-eye swimming goggle ( or the single-eye swimming goggle 3 ) be varied and posited via the adjustment step.

10. A method of wearing a swimming goggles comprising: a first step and a second step wearing a first and a second single-eye swimming goggle on periphery of a wearer's eye-holes, and an adjustment step of independently adjusting each single-eye swimming goggle in conformity with the periphery of eye-hole.
